(19) United States
(12) Patent Application Publication
(10) Pub. No.: US 2004/0064015 A1
(43) Pub. Date: Apr. 1, 2004

(54) Endoscope treatment-device and measuring method

(75) Inventors: Hiroaki Goto, Hachioji-shi (JP); Seiko Yunoki, Fussa-shi (JP); Kenji Shibaki, Hachioji-shi (JP)

Correspondence Address: SCULLY SCOTT MURPHY & PRESSER, PC, 400 GARDEN CITY PLAZA, GARDEN CITY, NY, 11530

(73) Assignee: OLYMPUS OPTICAL CO., LTD., TOKYO (JP)

(21) Appl. No.: 10/456,278

(22) Filed: Jun. 6, 2003

(30) Foreign Application Priority Data
Jun. 7, 2002 (JP) 2002-166901
Jun. 3, 2003 (JP) 2003-157734

(51) Int. Cl. A61B 1/00

(52) U.S. Cl. 600/104

(57) ABSTRACT

An endo-therapy accessory device used in combination with an endoscope having an endo-therapy accessory channel includes a catheter and an endo-therapy accessory inserted into the catheter. The catheter is inserted into the endo-therapy accessory channel of the endoscope, and has a tip opening, and a first main-index recognized at a hand side in a predetermined position of a rear side from the tip opening. The endo-therapy accessory is inserted into the catheter so that its tip is recessed/projected from the tip opening, and has a second main-index in a position where a distance from the tip coincides with a distance from the tip opening of the catheter to the first main-index, and an insertion portion of the endo-therapy accessory to recognize a projection length when projected from the tip opening of the catheter based on a relative position of the second main-index with the first main-index of the catheter.

### CROSS-REFERENCE TO RELATED APPLICATIONS

This application is based upon and claims the benefit of priority from the prior Japanese Patent Applications No. 2002-166901, filed Jun. 7, 2002; and No. 2003-157734, filed Jun. 3, 2003, the entire contents of which are incorporated herein by reference.

### BACKGROUND OF THE INVENTION

1. Field of the Invention

The present invention relates to an endo-therapy accessory device which is used in combination with an endoscope, and endo-therapy accessory projected length-recognition method.

2. Description of the Related Art

Generally, during a diagnosis or a treatment of a pancreatic bile duct, a plurality of endo-therapy accessories is used in combination. An endo-therapy accessory such as an endoscopic catheter is inserted through an endo-therapy accessory channel disposed in an insertion portion of an endoscope, and an endo-therapy accessory such as a guide wire is inserted into the catheter and used as an endo-therapy accessory device. During use of this endo-therapy accessory device, the insertion portion of the endoscope is first inserted into a part of a patient, e.g., a duodenum. Through the endo-therapy accessory channel of the endoscope inserted into the body, the catheter and the guide wire, i.e., the endo-therapy accessory device reaches a target portion to carry out a treatment/diagnosis.

When the catheter and the guide wire are operated, positions or movements of the tips thereof are confirmed by an endoscopic observation monitor. Generally, in a state where the insertion portion of the endoscope is inserted into the duodenum, the inside of the pancreatic bile duct is hidden behind of a papilla (narrowed area) which is an outlet to the duodenum. Accordingly, it is difficult to confirm the tips of the catheter and the guide wire from the distal end of the insertion portion of the endoscope arranged in the duodenum. Thus, for confirmation of the tip positions and the tip movements of the guide wire, the catheter etc. positioned in the pancreatic bile duct, imaging, using X-rays irradiated onto the patient, is used.

When such a treatment/diagnosis is carried out, the irradiation of the patient with X-rays imposes a load on the patient. Therefore, in order to confirm a position of the treatment/diagnosis, using as little X-ray irradiation as possible, a distance from the treatment/diagnosis position to the papilla is sometimes measured. There are known some guide wire technologies which can measure a projected length of a guide wire from a tip of an endo-therapy accessory channel of an insertion portion of an endoscope by monitor observation of the endoscope. For example, U.S. Pat. No. 5,084,022 and U.S. Pat. No. 5,379,779 disclose technologies in which markings are made on the guide wire.

The specification of U.S. Pat. No. 5,084,022 discloses a technique in which markings are made at equal intervals along a longitudinal direction of a guide wire. The markings are disposed in a streak shape in a circumferential direction of the guide wire. For the markings, the number of the streaks is gradually increased toward a hand side of the guide wire. As a distance from a tip of the guide wire to a position of the marking is beforehand known, the number of the marking streaks disposed in the guide wire is confirmed by a monitor of the endoscope to measure a distance from the tip of the guide wire. That is, when such a guide wire is used, the marking projected from a papilla to a proximate side is confirmed by the monitor of the endoscope in a state where the tip of the guide wire is arranged in a treatment/diagnosis position of a pancreatic bile duct. By using this monitor to confirm the number of marking streaks, a distance from the treatment/diagnosis position to the papilla can be measured.

The specification of U.S. Pat. No. 5,379,779 discloses a guide wire having radiopaque markings. The length of these markings can be measured by using radioactive rays such as X-rays, even if the marking cannot be observed through an endoscope. By using this guide wire, even when the inside of a papilla cannot be confirmed by the endoscopic monitor, the use of this guide wire enables a tip of the guide wire, the papilla and the radiopaque markings of the papilla portion on the guide wire to be confirmed on an X-ray observation monitor, thereby measuring the length.

### BRIEF SUMMARY OF THE INVENTION

According to an aspect of the present invention, an endo-therapy accessory device used in combination with an endoscope having an endo-therapy accessory channel to be used includes: a catheter which is inserted into the endo-therapy accessory channel of the endoscope, and which has a tip opening where a tip is opened, and a first main index recognized at a hand side in a predetermined position of a rear side from the tip opening; and an endo-therapy accessory which is inserted into the catheter so that its tip projects/goes down from/into the tip opening, and which has a second main index in a position where a distance from the tip coincides with a distance from the tip opening of the catheter to the first main index, and an insertion portion of the endo-therapy accessory to recognize a projection length when projected from the tip opening of the catheter based on a relative position with the first main index of the catheter.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic appearance view showing an endoscope where an endo-therapy accessory device of a first embodiment is arranged.

FIG. 2 is a schematic perspective view showing a state where a catheter and a guide wire of the endo-therapy accessory device of the first embodiment are combined.

FIG. 3 is a schematic perspective view showing a state where the catheter and the guide wire of the endo-therapy accessory device of the first embodiment are separated from each other.

FIG. 4A is a schematic side view of the guide wire in the endo-therapy accessory device of the first embodiment.

FIG. 4B is a vertical sectional view of the guide wire shown in FIG. 4A.

FIG. 4C is a transverse sectional view cut along the 4C-4C line shown in FIG. 4B.

FIG. 4D is a transverse sectional view cut along the 4D-4D line shown in FIG. 4B.

FIG. 5 is a schematic sectional view showing a state where a treatment is carried out by leading out the endo-therapy accessory device of the first embodiment through an endo-therapy accessory channel of the endoscope from a distal end of an insertion portion.

FIG. 6 is a schematic perspective view showing a modified example of the endo-therapy accessory device of the first embodiment in a state where the catheter and the guide wire are separated from each other.

FIG. 7A is a schematic vertical sectional view of an endo-therapy accessory device of a second embodiment.

FIG. 7B is a transverse sectional view cut along the 7B-7B line of FIG. 7A.

FIG. 8 is a schematic perspective view showing a state where a catheter and a guide wire of an endo-therapy accessory device of a third embodiment are separated from each other.

FIG. 9 is a schematic perspective view showing a state where a catheter and a guide wire of an endo-therapy accessory device of a fourth embodiment are separated from each other.

FIG. 10A is a schematic perspective view showing a state where a catheter and a guide wire of an endo-therapy accessory device of a fifth embodiment are separated from each other.

FIG. 10B is a schematic enlargement of a position indicated by a reference numeral 10B of FIG. 10A.

FIG. 11A is a schematic side view showing a modified example of the endo-therapy accessory device of the fifth embodiment where streak-shaped shape change sections are arranged at a predetermined interval before and after a reference index of a guide wire to be thicker more apart from the reference index.

FIG. 11B is a schematic side view of the guide wire showing a modified example of the endo-therapy accessory device of the fifth embodiment where a streak-shaped index and a shape change section which numerical value is smaller closer to the reference index are arranged at a predetermined interval before and after the reference index of the guide wire.

### DETAILED DESCRIPTION OF THE INVENTION

The embodiments of the present invention will be described with reference to the accompanying drawings.

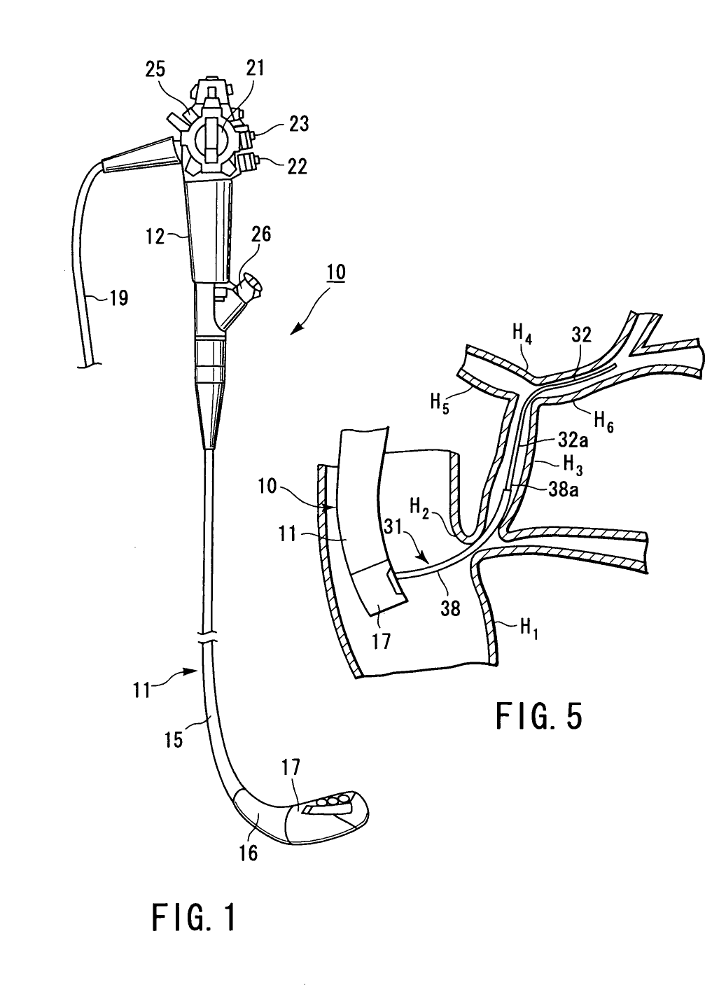

First, a first embodiment will be described by referring to FIGS. 1 to 5. An endoscope 10 interpolated in an endo-therapy accessory device 30 of the embodiment is constituted as shown in FIG. 1.

As shown in FIG. 1, the endoscope 10 includes a long, thin insertion portion 11 inserted into a body cavity, and an operation section 12 connected to a rear end of the insertion portion 11. The insertion portion 11 includes a long, thin tube portion 15 having flexibility, a bending portion 16 connected to a tip of the flexible tube portion 15, and a hard tip 17 arranged in an utmost tip position of the insertion portion 11. The insertion portion 11 includes an endo-therapy accessory channel through which an endo-therapy accessory is inserted, an illumination optical system for illuminating an object to be treated, and an observation optical system for observing the illuminated object to be treated. The observation optical system includes an objective lens in a tip 17 of the insertion portion 11, and the illumination optical system includes an illumination lens in the tip 17 of the insertion portion 11. In the observation optical system, for example, a not-shown CCD element is disposed to pick up an image of a light made incident on the objective lens. The CCD element is electrically connected to the operation section 12. In the insertion portion 11, preferably, an air/water channel or a suction channel is further disposed side by side with the endo-therapy accessory channel. The endoscope 10 of the embodiment is formed as a so-called side view type where an observation visual field direction of the objective lens in the tip 70 is in an inclined direction, for example, orthogonal to an axial direction of the insertion portion 11.

On the other hand, a rear end of a universal cord 19 is connected to the operation section 12 of the endoscope 10. In a tip of the universal cord 19, a light guide tube is disposed to guide a light to the illumination optical system, and an electrical contact section (not shown) is disposed to be electrically connected through the operation section 12 to the CCD element of the observation optical system. The light guide tube and the electrical contact section are respectively connected to a light source device and an image-processing device (not shown) which are external devices. That is, the light source device emits an illumination light, which is passed through the light guide tube and the illumination optical system to irradiate the object to be treated from the illumination lens. The object irradiated with the light is illuminated, and an image of the illumination light is picked up by the CCD element through the objective lens. The image picked up by the CCD element is transmitted from the observation optical system through the electrical contact section to the image-processing device. An endoscopic observation monitor (not shown) is connected to the image-processing device, and the endoscopic observation image picked up by the CCD element is displayed on the monitor.

In the operation section 12 of the endoscope 10, a bending operation section 21 for bending a bending portion 16 of the insertion portion 11 up-and-down and left-and-right by a remote control operation, an air/water supply button 22, and a suction operation button 23 are disposed. The air/water supply button 22 is used when the objective lens and the illumination lens disposed in the tip 17 of the air/water channel are washed to remove stains on the lenses, thereby facilitating visual recognition of an organ of a body to be treated (organism). The suction operation button 23 is used when blood or tissues accumulated during the treatment are discharged through the suction channel to the outside of the body.

The bending operation section 21 is arranged in the operation section 12 in a state where it is projected in a direction roughly orthogonal to a center axial direction of the insertion portion 11. In a position adjacent to this bending operation section 21, a elevator operation knob 25 is disposed to lift a later-described endo-therapy accessory elevator (not shown). Further, in the vicinity of the connection portion between the insertion portion 11 and the operation section 12, a biopsy valve (insertion port) 26 is arranged to be communicated with the endo-therapy accessory channel.

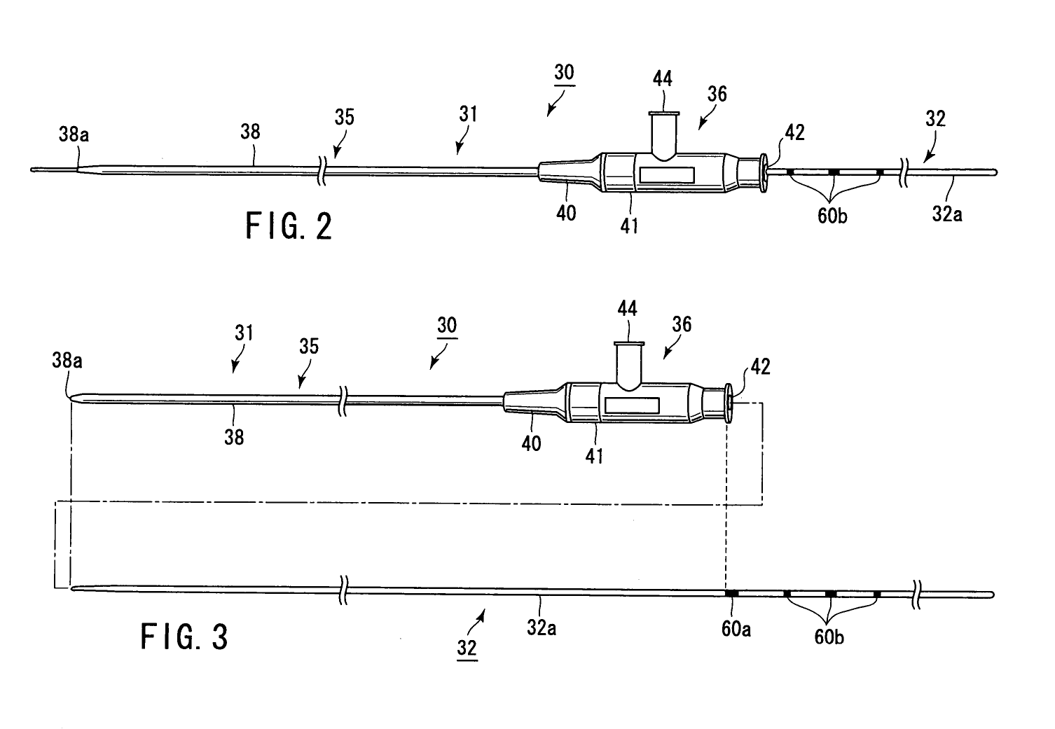

As shown in FIGS. 2 and 3, the endo-therapy accessory device 30 inserted through the endo-therapy accessory channel disposed in the insertion portion 11 of the endoscope 10 includes a catheter 31, and a guide wire 32 as an endo-therapy accessory interpolated in the catheter 31. This guide wire 32 must be inserted through the endo-therapy accessory channel even when the catheter 31 is pulled out from the endo-therapy accessory channel, and projected from the tip 17 of the insertion portion 11 of the endoscope 10. Thus, the guide wire 32 is formed to be longer than a combined length of a total length of the catheter 31 and a length of the endo-therapy accessory channel. For example, a total length of the catheter 31 is about 200 cm and, preferably, a total length of the guide wire 32 is about 400 cm.

The catheter 31 includes an insertion section (sheath insertion section) 35 inserted through the endo-therapy accessory channel, and a holding section (sheath holding section) 36 disposed in a rear end of the insertion section 35. The insertion section 35 of the catheter 31 has a long, thin tube-shaped sheath 38 having flexibility. The sheath 38 is made of an easily bent and soft material such as polyethylene or PTFE. Additionally, the sheath 38 is formed to be transparent or translucent so that the operator can visually recognize the inside when an endo-therapy accessory such as the guide wire 32 is inserted. An outer periphery of a tip of the sheath is preferably formed thinner compared with an outer periphery of a rear end so as to enter the narrowed area or the like.

The holding section 36 of the catheter 31 includes a sheath protection section 40 for covering the surroundings of the rear end of the sheath 38 to prevent bending, a cock 41 disposed in a rear end of the sheath protection section 40, and a rear end opening (opening end) 42 disposed in a rear end of the cock 41. A tip opening (first opening) 38a disposed in the tip of the sheath 38 and the rear end opening (second opening) 42 disposed in the rear end of the cock 41 are communicated with each other through a communication path. An injection port 44 is disposed in the cock 41 of the holding section 36. Since a not-shown liquid supply mechanism such as a syringe or a liquid supply tube is connected to the injection port 44, it is possible to supply a chemical solution, such as a contrast medium from the injection port 44, through the tip opening 38a of the sheath 38 to a desired portion of the body of the patient.

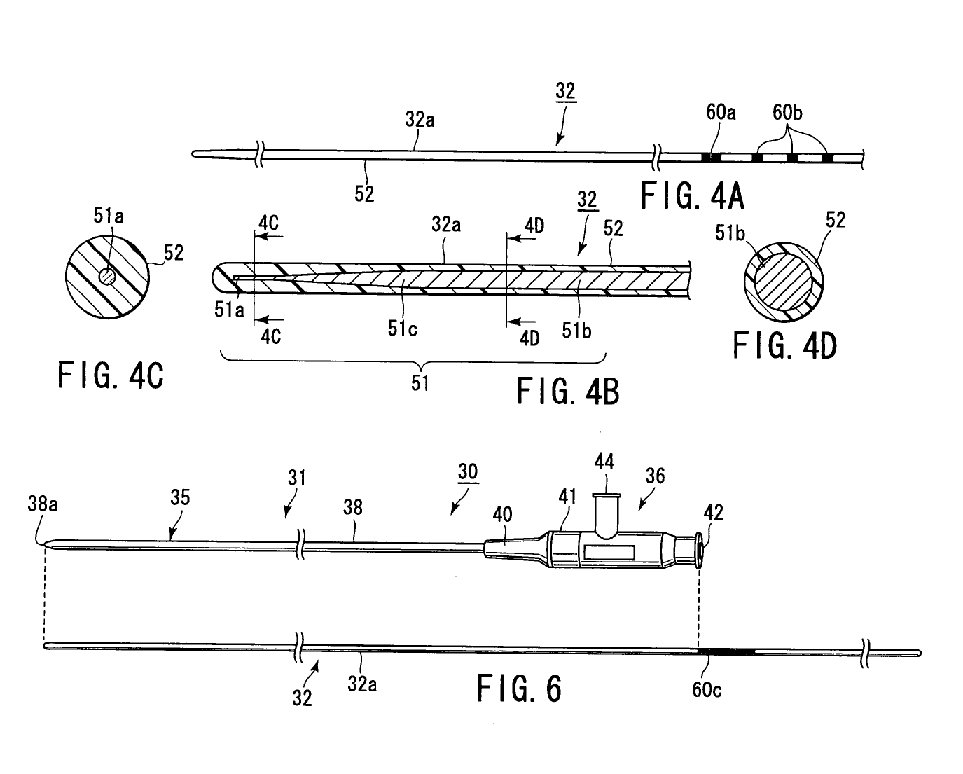

As shown in FIGS. 4A to 4D, the guide wire 32 is formed as a long, thin wire insertion section 32a as a whole. The insertion section 32a of the guide wire 32 includes a long and thin core material 51, and a cover 52 to cover an outer periphery of the core material 51.

As shown in FIGS. 4C and 4D, the core material 51 of the guide wire 32 has diameters different between a tip side and a center side. As shown in FIG. 4C, the tip side of the core material 51 is formed as a small diameter portion (small diameter core material) 51a. As shown in FIG. 4D, the center side of the core material 51 is formed as a large diameter portion (large diameter core material) 51b slightly larger in diameter than the small diameter portion 51a. As shown in FIG. 4B, the small diameter portion 51a and the large diameter portion 51b are integrally formed by a tapered portion 51c positioned therebetween. That is, the core material 51 having portions different in diameter is formed as one member, and formed integrally by the small diameter portion 51a, the large diameter portion 51b and the tapered portion 51c. In order to have flexibility, this core material 51 is made of, e.g., a super-elastic alloy, such as a nickel titanium alloy. For the core material 51, the small diameter portion 51a is formed to be higher in flexibility (ductility) than the large diameter portion 51b. That is, for the core material 51, the small diameter portion 51a is bent more easily than the large diameter portion 51b.

The cover 52 is made of, e.g., plastic materials such as polyurethane or PTFE formed, and the core material 51 is covered with this cover 52 without any space. Accordingly, when an external force is applied to the guide wire 32, the cover 52 is bent integrally with the core material 51 made of the superelastic alloy material.

Additionally, the cover 52 has, e.g., nonconductivity (insulation). An outer diameter of the cover 52 which covers the outer periphery of the large diameter portion 51b of the core material 51 is, e.g., about 1 mm. On the other hand, an outer diameter of the cover 52 which covers the outer periphery of the small diameter portion 51a of the core material 51 is, e.g., about 0.5 mm to 1 mm. Thus, since a maximum outer diameter of the guide wire 32 is about 1 mm, it is difficult to recognize a tip position of the guide wire 32 in the body using X-rays.

On the outer peripheral surface of the guide wire 32 having the foregoing constitution, a reference index (a second main index) 60a is disposed in a position on the cover 52 which coincides with a total length from the tip of the guide wire 32 to the catheter 31. That is, the reference index 60a coincides with the rear opening (index) 42 of the catheter 31 when the tips of both of the catheter 31 and the guide wire 32 coincide with each other. On a more rearward side from the reference index 60a of the guide wire 32, a plurality of indexes (subindexes) 60b are disposed at predetermined intervals, e.g., at 20 mm.

As such indexes 60a, 60b, for example, paint of a color different from that of the cover 52 is coated on the outer peripheral surface of the cover 52 of the guide wire 32. In addition, as the indexes 60a, 60b, preferably, a laser marking of a color different from that of the cover 52 is executed. As the indexes 60a, 60b, preferably, a material different from that of the cover 52, e.g., a heat-shrinkable tubing which is shrunk when heat is added thereto, is fixed on the outer peripheral surface of the cover 52 of the guide wire 32. The heat-shrinkable tubing used for each of the indexes 60a, 60b is preferably colored differently from the cover 52 of the guide wire 32. Each subindexes 60b is used for different materials and method of marking, and for same materials and same method of marking different from the colors of paint.

Then, when the guide wire 32 of FIG. 3 is inserted from the rear end opening 42 of the catheter 31, the endo-therapy accessory device 30 is formed as shown in FIG. 2, and the treatment device 30 is used in this state.

Next, description will be made of an operation of measuring a projected length of the tip of the guide wire 80 from the tip opening 38a of the catheter 90 by using the endo-therapy accessory device 30 of the foregoing constitution with reference to FIG. 5.

The tip 17 of the insertion portion 11 of the endoscope 10 is first inserted into, e.g., a portion near the papilla H2 of the duodenum H1. The tip of the catheter 31 is inserted from the biopsy valve 26 of the endoscope 10 through the endo-therapy accessory channel into the body of the patient.

The guide wire 32 is inserted from the rear end opening 42 of the catheter 31 toward the tip opening 38a. At this time, the indexes 60a, 60b of the guide wire 32 are still on a rear side (outside) more than the rear end opening 42 of the catheter 31.

The tip 38a of the sheath 38 of the catheter 31 is led out from the tip 17 of the insertion portion 11 of the endoscope 10, and the tip 38a of the sheath 38 of the catheter 31 is arranged near the papilla H2 while the observation monitor of the endoscope 10 is observed. When the tip 38a of the catheter 31 is projected from the tip 17 of the insertion portion 11 of the endoscope 10 in this way, the elevator operation knob 25 of the endoscope 10 is operated. The endo-therapy accessory base of the tip 17 of the insertion portion 11 is raised/lowered, and the tip 38a of the catheter 31 is inclined in an axial direction of the insertion portion 11 of the endoscope 10 to be guided to the outside.

Then, the catheter 31 is set in a retained state relative to the endo-therapy accessory channel of the insertion portion 11 of the endoscope 10. In this state, the tip of the guide wire 32 is led out from the tip of the catheter 31 toward the papilla H2 while the hand side is watched. At this time, by the indexes 60a, 60b of the hand side, a projected amount of the tip of the guide wire 32 from the tip opening 38a of the catheter 31 is recognized to be controlled. At this time, the reference index 60a enters the inside of the holding section 36 of the catheter 31, and thus it cannot be observed.

The tip of the guide wire 32 is inserted into/pulled out from the narrowed area of the papilla H2, and the guide wire 32 is rotated around an axis to be inserted into the deep side of the narrowed area of the papilla H2. The tip of the guide wire 32 is inserted from the papilla H2 of the patient into a common bile duct. The patient is irradiated with X-rays to observe slightly observed movement (position) of the tip of the guide wire 32 on the X-ray observation monitor. The tip of the guide wire 32 is inserted into one (target direction) of pipe lines H5, H6 branched at a porta hepatis H4, and the tip of the guide wire 32 is guided to a target portion (affected portion) in the body. The projected amount of the guide wire 32 from the tip opening 38a of the catheter 31 is recognized (visually recognized) by a plurality of indexes 60b disposed in parallel on the rear side of the catheter 31. That is, a projected length of the tip of the guide wire 32 from the tip opening 38a of the catheter 31 can be measured by the index 60b disposed on the rear side of the reference index 60a of the guide wire 32 in a state where the tip of the guide wire 32 is projected from the tip opening 38a of the catheter 31.

At this time, the tip opening 38a of the catheter 31 is arranged near the papilla H2. Thus, a distance from the target portion to the papilla H2 is substantially equal to the projected length of the guide wire 32 from the tip opening 38a of the catheter 31. Here, a holding position of the guide wire 32 is changed, e.g., gripping thereof near the rear end opening 42 of the catheter 31. At this time, instead of the gripping the guide wire 32, the guide wire 32 is marked by the pen.

The tip of the guide wire 32 is arranged in the target portion so as not to be moved, and the tip of the catheter 31 is similarly passed along the guide wire 32 through the papilla H2 of patient to be inserted into the common bile duct H3. Subsequently, the tip 38a of the catheter 31 is inserted along the guide wire 32 into one of the two pipe lines H5, H6 branched at the porta hepatic H4, and the tip 38a of the catheter 31 is guided to the target portion in the body.

At this time, the catheter 31 pushed into a position of the guide wire 32 where the reference index 60a on the guide wire 32 appears (coincides) in the rear end 42 of the catheter 31. Thus, the tip position of the guide wire 32 and the tip opening 38a of the catheter 31 are aligned with each other. Then, by measuring a distance from the holding changing position to grip the guide wire 32 to the reference index 60a of the guide wire 32 (rear end opening 42 of the catheter 31), it is possible to measure a distance from the target portion to the narrowed area of the papilla H2.

In this state, if a chemical solution such as the contrast medium is injected to the injection port 44 of the holding section 36 of the catheter 31, this chemical solution is applied from the holding section 36 through the tip opening 38a of the sheath 38 to the affected portion. That is, a treatment can be carried out for the affected portion by the catheter 31 (endo-therapy accessory).

As described above, the embodiment provides the following effects.

By using the rear end opening 42 of the catheter 31 as the index corresponding to the index 60a of the guide wire 32 to observe the rear end opening 42 of the catheter 31, it is possible to easily recognize the projected length of the tip of the guide wire 32 form the tip opening 38a of the catheter 31. That is, the projected length of the tip of the guide wire 32 from the tip opening 38a of the catheter 31 can be easily recognized by recognition at the hand side of the operator himself. Then, the use of the endoscope 10 and an X-ray observation monitor is minimized to confirm the position of the reference index 60a of the guide wire 32 with respect to the opening end (index) 42 of the catheter 31. In this way, it is possible to easily recognize the projected amount of the guide wire 32 from the tip opening 38a of the catheter 31. That is, without observing the endoscope 10 or the X-ray observation monitor, the projected length of the guide wire 32 from the tip 38a of the catheter 31 can be measured at the hand side of the operator. Therefore, it is possible to reduce a load on the patient, such as long irradiation of the patient with X-rays.

Incidentally, according to the embodiment, the indexes 60b are disposed at predetermined intervals in the rear end of the reference indexes 60a and, by such an index 60b, the projected length of the tip of the guide wire 32 from the tip opening 38a of the catheter 31 can be measured. In addition, as shown in FIG. 6, a reference index 60c may be disposed longer along the axial direction than the reference index 60a shown in FIG. 3 and FIG. 4A. Thus, the operator finds the reference index 60c more easily, and can read it more easily. Moreover, a plurality of indexes 60b may be arranged side by side at predetermined intervals on a more rearward side than the reference index 60c.

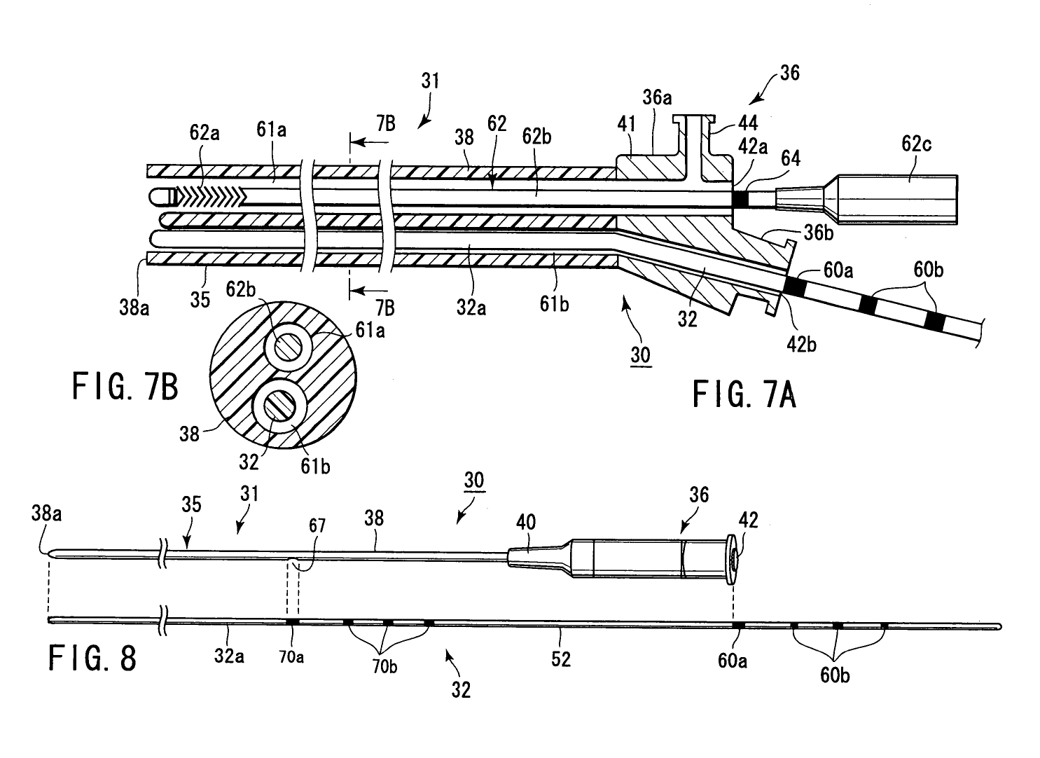

Next, a second embodiment will be described by referring to FIGS. 7A and 7B. This embodiment is a modified example of the first embodiment. Thus, members similar to those of the first embodiment will be denoted by similar reference numerals, and a detailed description thereof will be omitted.

As shown in FIGS. 7A and 7B, two lumens (first and second lumens) 61a, 61b are formed in the catheter 31 of the endo-therapy accessory device 30 of the embodiment. As shown in FIG. 7A, a holding section 36 of the catheter 31 is divided into two corresponding to the lumens 61a, 61b to form first and second holding sections 36a, 36b. The first holding section 36a is disposed on the rear side of the first lumen 61a along a center axis of the sheath 38. In a rear end of the first holding section 36a, a first rear end opening (opening end) 42a is formed to be communicated with the first lumen 61a.

An endo-therapy accessory such as a cytodiagnosis brush 62 is inserted into the first lumen 61a of the catheter 31, and the guide wire 32 is inserted into the second lumen 61b. As shown in FIG. 7A, the cytodiagnosis brush 62 inserted into the first lumen 61a includes a shaft-shaped brush insertion section 62b having a brush (treatment section) 62a on its tip, and a brush holding section 62c connected to a rear end of the brush insertion section 62b. An opening diameter of the first rear end opening 42a of the first holding section 36a is smaller compared with an outer diameter of the brush holding section 62c of the cytodiagnosis brush 62. Thus, insertion of the holding section 62c into the first lumen 61a is prevented.

The brush insertion section 62b of the cytodiagnosis brush 62 has an index 64 on an outer peripheral surface corresponding to the first rear end opening 42a of the catheter 31 when a tip of the brush 62a (tip of the brush insertion section 62b) is aligned with the tip opening 38a of the catheter 31.

On the other hand, a second holding section 36b of the catheter 31 is disposed in a direction where it is bent by a predetermined bending angle in an axial direction of a center axis of the sheath 38. In a rear end of the holding section 36b, a second rear end opening (opening end) 42b is formed to be communicated with a lumen of the sheath 38.

Next, description will be given of an operation of measuring projected lengths of tips of a guide wire 32 and the cytodiagnosis brush 62 from a tip end opening 38a of a catheter 31 by using the endo-therapy accessory device 30 of the foregoing constitution.

First, as described above with reference to the first embodiment, the guide wire 32 is inserted into the second lumen 61b of the catheter 31 projected from the tip 17 of the endoscope 10, and the tip of the guide wire 32 reaches the target portion. Then, the tip opening 38a of the catheter 31 is aligned with the tip of the guide wire 32 to carry out a treatment such as length measurement.

In this state, the cytodiagnosis brush 62 is inserted into the empty first lumen 61a of the catheter 31, and the index 64 of the cytodiagnosis brush 62 is matched with the first rear end opening 42a of the catheter 31. At this time, the tip of the brush 62a coincides with the tip opening 38a of the catheter 31. After the index 64 of the cytodiagnosis brush 62 has been matched with the first rear end opening 42a of the catheter 31, the catheter 31 is slightly pulled out with respect to the cytodiagnosis brush 62. Measurement of a pulling amount at this time is carried out by using the index 60b of the rear side with respect to the reference index 60a of the guide wire 32. Alternatively, it is pulled out until the first opening end 42a is abutted on a brush holding section 62c. Then, the brush 62a of the tip of the cytodiagnosis brush 62 is exposed to touch the affected portion.

The holding section 62c of the cytodiagnosis brush 62 is operated to collect tissues of the affected portion. The cytodiagnosis brush 62 is completely pulled out from the first lumen 61a of the catheter 31 to inspect the collected tissues.

Then, the catheter 31 is inserted along the guide wire 32 until a second rear end opening 42b of the catheter 31 coincides with the index 60a of the guide wire 32. A new endo-therapy accessory is inserted into the empty first lumen 61a to carry out a similar treatment.

As the endo-therapy accessory, in addition to the cytodiagnosis brush 62 shown in FIG. 7A, various endo-therapy accessories such as a gripping forceps, a biopsy forceps, an injection needle, an imaging tube, a stone crusher, a clipper, a high-frequency snare, a papillotomy knife etc., can be used. That is, when the tip of the endo-therapy accessory is matched with the tip of the catheter 31, the index 64 is set in a position coincident with the rear end opening 42 of the catheter 31. Then, in a state where the guide wire 32 is not moved from the target portion, by matching the tip opening 38a of the catheter 31 with the tip of the guide wire 32, it is possible to easily guide the treatment section of the tip of the endo-therapy accessory to the affected portion.

Therefore, the treatment can be carried out without searching the position of the affected portion each time the endo-therapy accessory is removed from the first lumen 61a of the catheter 31 to insert a new treatment into the first lumen 61a of the catheter 31. That is, general applicability of the endo-therapy accessories can be greatly improved.

The embodiment has been described by way of example where the tip of the endo-therapy accessory is matched with the tip opening 38a of the catheter 31. Depending on an endo-therapy accessory, since it needs to be arranged slightly more on the rear end side than the tip opening 38a of the catheter 31, it is not necessary to completely match the tip of the endo-therapy accessory with the tip opening 38a of the catheter 31. That is, when the reference index disposed in the endo-therapy accessory coincides with the rear end opening 42a of the catheter 31, the case of arrangement of the tip of the endo-therapy accessory on a more proper distance rear side than the tip opening 38a of the catheter 31 is also within the coincidence with the tip opening 39a of the catheter 31.

Next, a third embodiment will be described with reference to FIG. 8. This embodiment is a modified example of the first embodiment. Thus, members similar to those of the first embodiment are denoted by similar reference numerals, and detailed description thereof will be omitted.

For example, if a length of the insertion portion 11 (endo-therapy accessory channel) of the endoscope 10 is considerably shorter compared with that of the catheter 31, the rear end side of the catheter 31 may be greatly projected from the biopsy valve 26 of the endo-therapy accessory channel to the outside. In such a case, confirmation of the rear end opening 42 of the catheter 31 is very burdensome for the operator.

A total length of the catheter 31 of the endo-therapy accessory device 30 of the embodiment is considerably longer compared with that of the insertion portion 11 (endo-therapy accessory channel) of the endoscope 10. The injection port 44 is removed from the cock 41 of the holding section 36 of the catheter 31. The sheath 38 of the catheter 31 is not transparent, for example, and thus the guide wire 32 cannot be observed through. On the rear side of the sheath 38 of the catheter 31, a side hole (index) 67 is disposed as an observation window. The tip opening (first opening) 38a of the sheath 38, the rear end opening 42 of the sheath 38, and the side hole (second opening) 67 are communicated with one another.

On the other hand, in the guide wire 32, in addition to the reference index (first reference index) 60a described above with reference to the first embodiment, a second reference index (main index) 70a is disposed in a position corresponding to the side hole 67. The second reference index 70a coincides with the side hole 67 of the catheter 31 when the tip of the catheter 31 is matched with the tip of the guide wire 32. Preferably, in the guide wire, in addition to the index 60b on the rear side of the first reference index 60a, a plurality of indexes (subindexes) 70b are disposed at predetermined intervals, e.g., 20 mm on the rear side of the second reference index 70a.

Thus, when the second reference index 70a of the guide wire 32 is matched with the side hole 67 of the catheter 31, a projected amount of the tip of the guide wire 32 from the tip opening 38a of the catheter 31 can be recognized. At this time, if the second reference index 70a of the guide wire 32 is matched with the side hole 67 of the catheter 31, the first reference index 60a is matched with the opening end 42 of the catheter 31. Then, the side hole 67 is disposed in a position to allow easy observation by the operator, and the index 70a is disposed in a position corresponding to the side hole 67 of the guide wire 32, whereby the projected length of the tip of the guide wire 32 can be measured in the position to allow easy observation by the operator.

The embodiment shows the constitution where the side hole 67 is disposed in the catheter 31. However, this side hole 67 is covered with a transparent member to enable supplying of liquid from the holding section 36 (rear end opening 42) to the tip opening 38a.

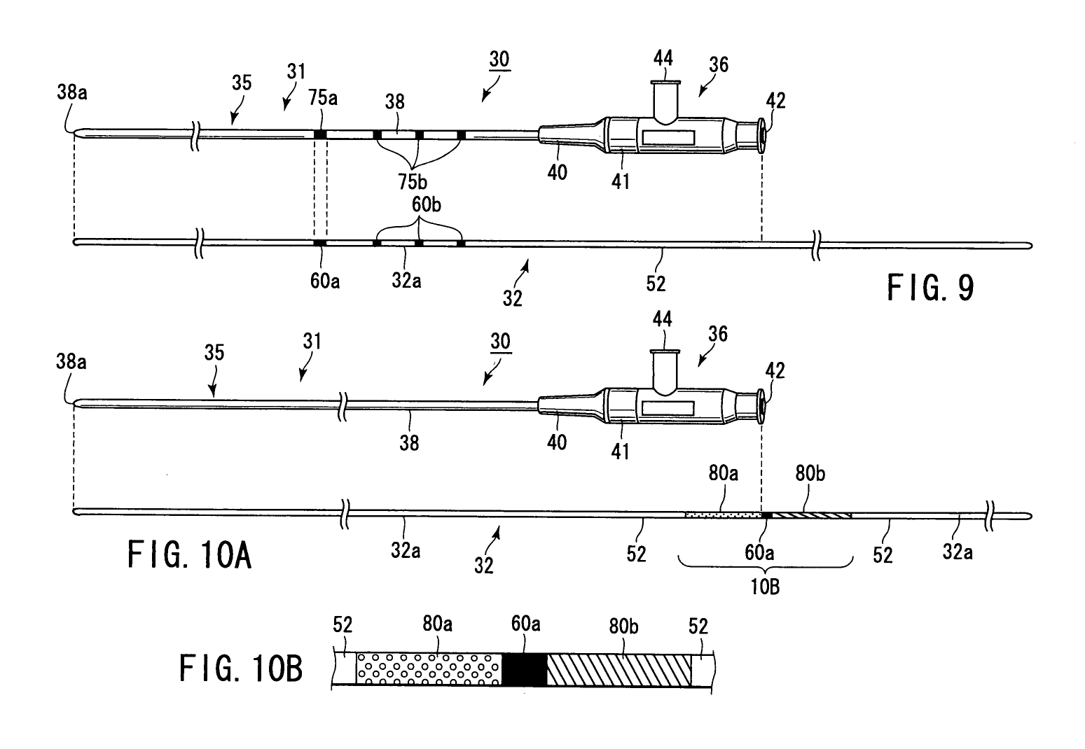

Next, a fourth embodiment will be described with reference to FIG. 9. This embodiment is a modified example of the first embodiment. Thus, members similar to those of the first embodiment are denoted by similar reference numerals, and detailed description thereof will be omitted.

A total length of the catheter 31 of the endo-therapy accessory device 30 is different from that from the tip of the guide wire 32 to the reference index 60a. This is a main difference between the catheter 31 of the first embodiment and the catheter 31 of this embodiment. That is, the sheath 38 of the insertion is section 35 of the catheter 31 is formed to be transparent (half transparent) so that it can be visually recognized when the guide wire 32 is inserted.

According to the embodiment, a reference index (a first main index) 75a is disposed from the tip opening 38a of the catheter 31 to the sheath 38 of the rear side so as to coincide with a distance from the tip of the guide wire 32 to the reference index 60a. In the sheath 38 on a more rearward side than the reference index 75a disposed in the sheath 38 of the catheter 31, a plurality of indexes (subindexes) 75b is disposed at predetermined intervals, e.g., 20 mm intervals. As in the case of the guide wire 32 of the aforementioned first embodiment, preferably, the indexes 75a, 75b disposed in the catheter 31 are colored differently from other portions, and covered with other materials to be easily identified by the operator. Preferably, the indexes 75a, 75b are disposed on the outer peripheral surface of the sheath 38. In addition, a thin index layer is preferably formed between the outer peripheral surface and the inner peripheral surface.

Therefore, if the rear end opening 42 of the catheter 31 of the first embodiment is replaced by the index 75a, an effect similar to that of the first embodiment is obtained. That is, the reference index 60a of the catheter 31 and the reference index 75a of the guide wire 32 in the catheter 31 are matched with each other by observing the guide wire 32 through the catheter 31. Then, a length from the papilla H2 to the target portion is measured.

According to the embodiment, the following effects can be obtained in addition to those of the first embodiment.

Irrespective of the length of the catheter 31, the reference indexes 60a, 75a are disposed at equal distances from the tips of the catheter 31 and the guide wire 32. That is, the reference index 75a is disposed in the sheath 38 of the catheter 31 to coincide with the reference index 60a of the guide wire 32. These reference indexes 60a, 75a are matched with each other by relatively moving the catheter 31 and the guide wire 32 back and forth. Then, the projected length of the guide wire 32 from the tip opening 38a of the catheter 31 can be measured at the hand side of the biopsy valve 26 of the endoscope 10.

As in the case of the first embodiment, when the tip opening 38a of the catheter 31 and the tip of the guide wire 32 are matched with each other, an index may be further disposed in a position which coincides with the length to the rear end opening 42 of the catheter 31. Then, it is possible to know the projected length by both of the reference index 75a of the catheter 31 and the rear end opening 42. That is, length measurement can be carried out even when the catheter 31 is inserted through the endo-therapy accessory channel of the endoscope 10 into the rear end side.

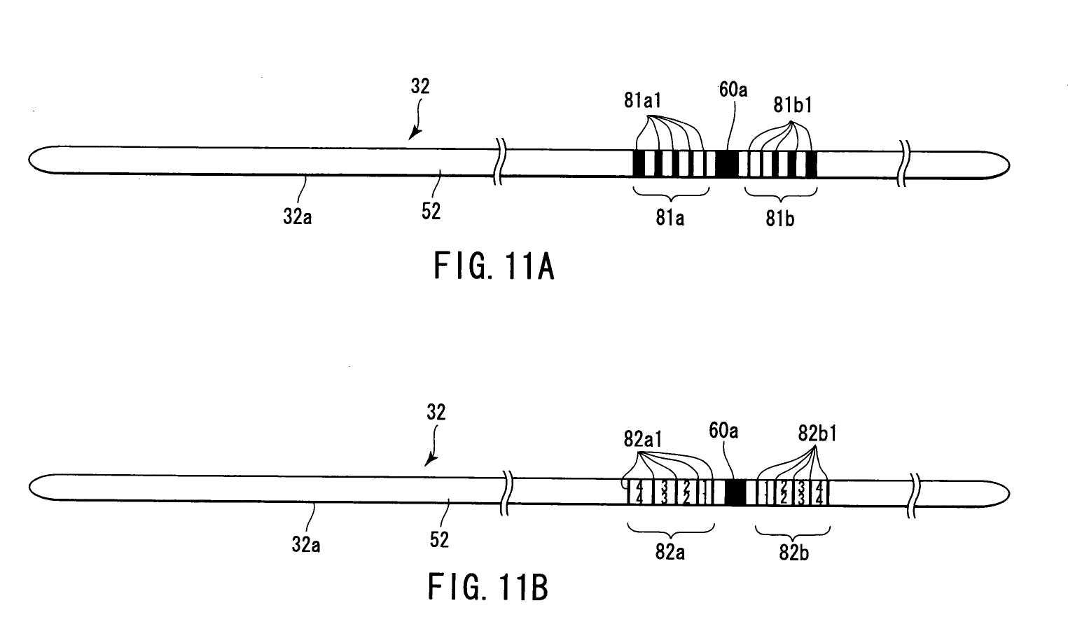

Next, a fifth embodiment will be described with reference to FIGS. 10A and 10B and FIGS. 11A and 11B. This embodiment is a modified example of the first embodiment. Thus, members similar to those of the first embodiment are denoted by similar reference numerals, and detailed description will be omitted.

As shown in FIGS. 10A and 10B, a first shape change section 80a where a number of dimples are formed is disposed, e.g., 5 cm on a front side adjacent to the reference index 60a of the guide wire 32. A second spiral shape change section 80b is formed, e.g. 5 cm on a rear side adjacent to the reference index 60a of the guide wire 32. These first and second shape change sections 80a, 80b are different in touch (hand feeling) from the cover 52 of the guide wire 32.

Thus, when the operator touches the first and second shape change sections 80a, 80b, the sensation received is different to that felt when touching the other portion (cover 52) of the guide wire 32. Then, when the operator grips a 5 cm range before/after the reference index 60a of the guide wire 32, the position of the reference index 60a is easily determined by a hand feeling. Thus, without observing the hand side of the catheter 31, the operator can easily match the reference index 60a and the rear end opening 42 of the catheter 31 with each other. As a result, the operator can measure the projected length of the guide wire 32 by moving the catheter 31 to the guide wire 32 while observing the other portions.

In the embodiment, the dimples and the spiral shape have been descried as the first and second shape change sections 80a, 80b. Other shape change sections, for example, recessed and projected forms or a reverse spiral direction may be employed. That is, it is only required of the operator to recognize, by touch, changes between the cover 52 and the shape change sections 80a, 80b before/after the reference index 60a when gripping the guide wire 32.

Additionally, as shown in FIGS. 11A and 11B, indexes may be disposed before/after the reference index 60a of the guide wire 32. As shown in FIG. 11A, a plurality of streak-shaped first and second shape change sections 81a, 81b are disposed side by side at predetermined intervals on the outer periphery orthogonal to the axial direction of the guide wire 32. These shape change sections 81a, 81b are formed such that streak-shaped indexes (subindexes) 81a1, 81b1 are thinner closer to the reference index 60a, and thicker more apart therefrom. Accordingly, even if the reference index 60a is hidden inside the holding section 36 of the catheter 31, the position of the reference position 60a can be easily known by visual observation. The second spiral shape change section 80b shown in FIGS. 10A and 10B has a similar effect. That is, even if the reference index 60a is hidden inside the holding section 36 of the catheter 31, by visually observing the second shape change section 80b, the position of the reference index 60a can be easily known.

Furthermore, as shown in FIG. 11B, preferably, for example, shape change sections 82a, 82b to which numerals (codes) 1 to 4 are allocated are disposed before/after the reference index 60a of the guide wire 32. For these numerals, numeral 1 is set proximate to the reference index 60a and, in a furthest apart position, e.g., numeral 4 is given. Between the adjacent numerals e.g., 1 cm is set. That is, the numerals 1 to 4 have an interval of 3 cm. Moreover, the numerals 1 to 4 may be held between the streak-shaped indexes 82a1, 82b1 before/after the reference index 60a of the guide wire 32. Then, the shape change sections 82a, 82b can be used as scales to the reference index 60a.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endo-therapy accessory device used in combination with an endoscope having an endo-therapy accessory channel, comprising:
a catheter which is inserted into the endo-therapy accessory channel of the endoscope, and which has a tip opening and a first main index recognized at a hand side in a predetermined position of a rear side from the tip opening; and
an endo-therapy accessory which is inserted into the catheter to project/go down from/into the tip opening of the catheter, and which has a second main index in a position where a distance from the tip coincides with a distance from the tip opening of the catheter to the first main index, and an insertion portion of the endo-therapy accessory to recognize a projection length when projected from the tip opening of the catheter based on a relative position of the second main index with the first main index of the catheter.

2. The endo-therapy accessory device according to claim 1, wherein the insertion portion of the endo-therapy accessory further has a plurality of subindexes on a rear side of the second main index.

3. The endo-therapy accessory device according to claim 1, wherein a total length of the catheter coincides with a distance from a tip of the insertion portion of the endo-therapy accessory to the second main index of the insertion portion of the endo-therapy accessory, and the first main index of the catheter is set as a rear end of the catheter.

4. The endo-therapy accessory device according to claim 3, wherein the insertion portion of the endo-therapy accessory further has a plurality of subindexes on a rear side of the second main index.

5. The endo-therapy accessory device according to claim 1, wherein in a position adjacent to the second main index, a shape changing section is formed, which has a shape different from the vicinity of the second main index and which shape change is recognized by touching of an operator.

6. The endo-therapy accessory device according to claim 1, wherein the catheter has at least two lumens, one lumen is inserted into the insertion portion of the endo-therapy accessory, and the second endo-therapy accessory is inserted into an empty lumen.

7. The endo-therapy accessory device according to claim 6, wherein the insertion section of the second endo-therapy accessory comprises a main index inserted into the empty lumen of the catheter so that a tip thereof projects/goes down from/into the tip opening of the catheter in a position where a distance from the tip coincides with a distance from the tip opening of the catheter to the first main index, and recognizes a projection length when projected from the tip opening of the catheter based on a relative position of the main index with the first main index of the catheter.

8. A measuring method which measures a length of a predetermined portion in a body cavity, including:
preparing the endo-therapy accessory device of claim 1,
advancing a tip of the catheter to a base end of the predetermined portion through the endoscope,
projecting the endo-therapy accessory from the tip of the catheter to position a tip of the endo-therapy accessory at a far end of the predetermined portion; and
measuring a distance between the main index of the endo-therapy accessory and the main index of the catheter.

9. A measuring method which measures a length of a predetermined portion in a body cavity, including:
preparing the endo-therapy accessory device of claim 3,
advancing a tip of the catheter to a base end of the predetermined portion through the endoscope,
projecting the endo-therapy accessory from the tip of the catheter to position a tip of the endo-therapy accessory at a far end of the predetermined portion;
marking a position of the insertion portion of the endo-therapy accessory which overlaps a position of the main index of the catheter; and
measuring a distance between the main index of the endo-therapy accessory and the marked position.
